(19) United States
(12) Patent Application Publication
(10) Pub. No.: US 2026/0218001 A1
(43) Pub. Date: Jul. 30, 2026

(54) (METH)ACRYLATED ISOCYANURATE MIXTURE, PROCESS FOR PREPARING SAME, AND USES THEREOF

(71) Applicant: Arkema France, Puteaux (FR)

(72) Inventors: Philippe CICERON, Senlis (FR); Xavier DRUJON, Verneuil En Halatte (FR); Alice LEFORT, Verneuil En Halatte (FR); Elodie ROBIN, Verneuil En Halatte (FR)

(21) Appl. No.: 19/143,937

(22) PCT Filed: Dec. 15, 2023
(86) PCT No.: PCT/EP2023/086025

§ 371 Date: Jun. 27, 2025

(30) Foreign Application Priority Data
Dec. 29, 2022 (FR) FR22.14645

(51) Int. Cl. C09D 135/02; B33Y 10/00; B33Y 70/00; C07D 251/32; C08F 222/22

(52) U.S. Cl. CPC C09D 135/02; B33Y 70/00; C07D 251/32; C08F 222/22; B33Y 10/00

(57) ABSTRACT

The present invention relates to a mixture of (meth)acrylated isocyanurates, to a process for preparing same, to a polymerizable composition comprising same, and to uses thereof, especially as binder in a polymerizable composition or in a composition for additive manufacturing, in particular for printing a 3D or 4D article.

### SUBJECT MATTER OF THE INVENTION

The present invention relates to a mixture of (meth)acrylated isocyanurates, to a process for preparing same, to a polymerizable composition comprising same, and to uses thereof, especially as binder in a polymerizable composition or in a composition for additive manufacturing, in particular for printing a 3D or 4D article.

### BACKGROUND OF THE INVENTION

Photocrosslinkable resins based on monomers and/or oligomers functionalized with (meth)acrylate groups are used especially in the manufacture of components by 3D printing, of coatings for various uses (especially in the field of graphic arts), of adhesives, and of sealants. These resins react under UV and/or LED energy to give the end product properties of hardness, flexibility and/or resistance to chemicals, water or stains. An acrylate monomer of this type is marketed by Arkema (Sartomer) under the reference SR368®. It consists of tris(2-hydroxyethyl) isocyanurate triacrylate (THEICTA). This monomer has outstanding reactivity, hardness, and chemical resistance properties, which give it an advantage over other polyol polyacrylate monomers, in particular trimethylolpropane triacrylate (TMPTA), while retaining the flexibility properties, solvent resistance, and stain resistance of materials obtained from TMPTA. THEICTA does however have the drawback of being solid, which means it must be heated to above 52-53° C. before use by formulators.

Polyester (meth)acrylates based on tris(2-hydroxyethyl) isocyanurate (THEIC) have moreover been described in patent JP94081782 (JPH0681782). These polymers are obtained by a two-step process, comprising a first step of partial (meth)acrylation of a mixture of polyols, inter alia THEIC and trimethylolpropane (TMP), followed by a step of polycondensation with a polyacid, inter alia adipic acid or tetrahydrophthalic anhydride, to form a polyester. The ratio of diacid to polyol(s) to (meth)acrylic acid is 1/2/4 or 1/2/2 and the ratio of THEIC to other polyol (in particular TMP) ranges from 1/3 to 3/1. The inclusion of THEIC monomer makes it possible to limit the inhibition of polymerization by atmospheric oxygen without using additives such as amines. The present inventors have found that the polymer obtained in this patent had a content of residual THEICTA amounting to less than 10% of the weight of the copolymer, plus TMPTA derived from TMP.

In the same vein, patent application CN101838377 discloses a photopolymerizable composition component obtained from a polyol (such as THEIC or pentaerythritol), a polybasic acid such as adipic acid, and (meth)acrylic acid, in a molar ratio of acid to polyol to (meth)acrylic acid of 1/2/7 in the case of a diacid.

However, it was observed that certain THEIC-based mono- and polyester (meth)acrylates had a tendency to grain formation at room temperature due to recrystallization of THEICTA. The formulators of these resins were therefore forced to heat them above the melting point of these crystals.

There therefore remains a need for a resin based on (meth)acrylated isocyanurates that does not exhibit any problems of recrystallization after at least four months at room temperature, while retaining the advantageous properties of THEICTA described above.

After intensive research, the applicant has developed a resin that makes it possible to meet the above requirement, as well as a process for preparing said resin with which it is possible to introduce specific impurities in a given amount into a tris(hydroxyalkyl) isocyanurate tri(meth)acrylate (THAICT(M)A). The product obtained is a mixture comprising predominantly THAICT(M)A, as well as condensation products of THAIC (meth)acrylated with a dicarboxylic acid to form a polyester. This product being a liquid, it is readily employable by formulators in 3D or additive manufacturing or in the manufacture of coatings or adhesives. In addition, it does not exhibit recrystallization for at least 4 months at ambient temperature after dilution in monomers with high diluting power (such as hexane-1,6-diol diacrylate) or in viscous monomers with a high Tg (such as tricyclodecanedimethanol diacrylate) used in these applications. The formulator thus has great flexibility in setting the dilution ratio, depending on the Tg and viscosity that he/she wishes to impart to the photopolymerizable composition. The compositions thus obtained also exhibit good reactivity, while the products obtained from these compositions exhibit excellent properties in terms of hardness, flexibility, and stain resistance.

### SUMMARY OF THE INVENTION

The invention relates to a mixture of (meth)acrylated isocyanurates, characterized in that it is obtained by reaction between:
(a) at least one dicarboxylic acid,
(b) at least one (meth)acrylic monomer selected from acrylic acid, methacrylic acid, anhydrides thereof, and mixtures thereof, and optionally at least one C₆-C₂₄ monocarboxylic acid,
(c) at least one tris(hydroxyalkyl) isocyanurate and optionally another polyol, it being understood that the molar ratio of —COOH groups of constituent (a) to —OH groups of constituent (c) is between 1:4 and 1:20, preferably between 1:5 and 1:15, more preferably between 1:6 and 1:12.

It also relates to a process for preparing the mixture of (meth)acrylated isocyanurates as described above, characterized in that it comprises the following steps:
1) reacting all or part of a constituent (b) comprising at least one (meth)acrylic monomer selected from acrylic acid, methacrylic acid, anhydrides thereof, and mixtures thereof and optionally at least one C₆-C₂₄ monocarboxylic acid with a constituent (c) comprising at least one tris(hydroxyalkyl) isocyanurate and optionally another polyol under conditions that permit the esterification of from 50 to 95%, preferably from 60 to 90%, more preferably from 70 to 85%, of the OH groups of constituent (c),
2) reacting the product of step 1) with a constituent (a) comprising at least one dicarboxylic acid and optionally with any residual amount of constituent (b).

The invention also provides a polymerizable composition characterized in that it comprises at least a mixture of (meth)acrylated isocyanurates as described above and at least one other ethylenically unsaturated compound, in particular a (meth)acrylate-functionalized monomer.

It also provides for the use of a mixture of (meth)acrylated isocyanurates as described above as a binder in a polymerizable composition or in a composition for additive manufacturing, in particular for printing a 3D or 4D article.

### DETAILED DESCRIPTION

In the remainder of this description, the expression “between” is understood as denoting a range of values that is inclusive of the stated limits.

The invention relates to a particular mixture of (meth)acrylated isocyanurates. For the purposes of the invention, the term “(meth)acrylated isocyanurate” corresponds to a compound having at least one isocyanurate group and at least one (meth)acrylate group.

As used herein, the term “isocyanurate” corresponds to a group of formula (I) below:

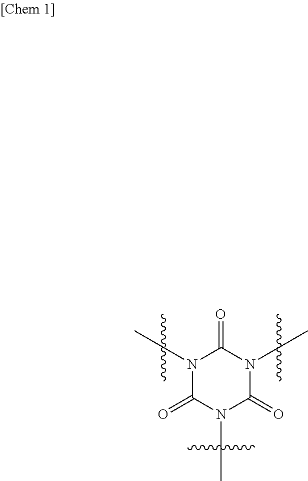 [SMILES: *n1c(=O)n(*)c(=O)n(*)c1=O]

As used herein, the term “(meth)acrylate group” means interchangeably an acrylate group (also termed acryloyloxy, of formula —O—CO—CH═CH₂) or a methacrylate group (also termed methacryloyloxy, of formula —O—CO—C(CH₃)═CH₂).

The mixture of the invention may in particular comprise a (meth)acrylated tris(hydroxyalkyl) isocyanurate and a (meth)acrylated polyester based on tris(hydroxyalkyl) isocyanurate.

For the purposes of the invention, the term “tris(hydroxyalkyl) isocyanurate”, or “THAIC”, means a compound corresponding to formula (II) below:

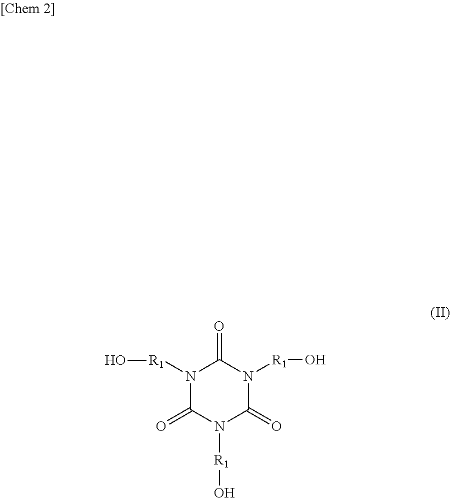

in which each R₁ is independently an optionally alkoxylated C₂-C₁₂ alkylene.

In particular, the THAIC may correspond to formula (II) in which each group R₁ is an ethylene (—CH₂—CH₂—). In this case, the THAIC is a tris(hydroxyethyl) isocyanurate, or THEIC.

For the purposes of the invention, the term “(meth)acrylated tris(hydroxyalkyl) isocyanurate”, or “(meth)acrylated THAIC”, corresponds to a tris(hydroxyalkyl) isocyanurate (THAIC) in which at least one of the OH groups has been converted into a (meth)acrylate group (i.e. by esterification with (meth)acrylic acid or a (meth)acrylic acid derivative). A (meth)acrylated THAIC may in particular comprise one or more compounds selected from a mono-, di- or tri(meth)acrylate of a tris(hydroxyalkyl) isocyanurate. These compounds may in particular correspond to formula (III) below:

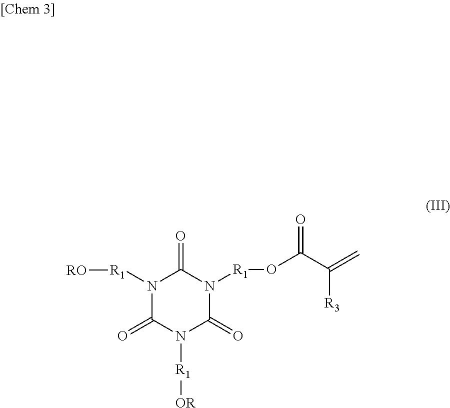

in which:
each R is independently H or a (meth)acryloyl group of formula —CO—C(R₃)═CH₂;
each R₁ is independently an optionally alkoxylated C₂-C₁₂ alkylene;
each R₃ is independently H or methyl.

In particular, the (meth)acrylated THAIC may correspond to formula (III) in which each group R₁ is an ethylene (—CH₂—CH₂—). In this case, the (meth)acrylated THAIC is a (meth)acrylated tris(hydroxyethyl) isocyanurate, or (meth)acrylated THEIC.

A compound of formula (III) in which each group R is a (meth)acryloyl group of formula —CO—C(R₃)═CH, is a tris(hydroxyalkyl) isocyanurate tri(meth)acrylate, or THAICT(M)A.

A compound of formula (III) in which each group R₁ is an ethylene (—CH₂—CH₂—) and each group R is a (meth)acryloyl group of formula —CO—C(R₃)—CH₂ is a tris(hydroxyethyl) isocyanurate tri(meth)acrylate, or THEICT(M)A.

For the purposes of the invention, the term “polyester” corresponds to a polymer molecule that includes at least two ester linkages. A polyester may consist of identical and/or different monomer units, preferably from 2 to 50, and more preferably from 2 to 10, identical and/or different monomer units, obtained by a polycondensation between at least one polyacid (or polycarboxylic acid) and at least one polyol. For the purposes of the invention, the term “(meth)acrylated polyester” corresponds to a polyester functionalized with at least one (meth)acrylate group. For the purposes of the invention, the term “tris(hydroxyalkyl) isocyanurate-based (meth)acrylated polyester” corresponds to a (meth)acrylated polyester incorporating monomer units derived from a THAIC and/or a (meth)acrylated THAIC. In particular, the tris(hydroxyalkyl) isocyanurate-based (meth)acrylated polyester may be a tris(hydroxyethyl) isocyanurate-based (meth)acrylated polyester, i.e. a (meth)acrylated polyester incorporating monomer units derived from a THEIC and/or a (meth)acrylated THEIC.

The mixture of (meth)acrylated isocyanurates of the invention is obtained specifically by reaction between:
(a) at least one dicarboxylic acid,
(b) at least one (meth)acrylic monomer and optionally at least one monocarboxylic acid,
(c) at least one tris(hydroxyalkyl) isocyanurate and optionally another polyol.

The various constituents of the reaction mixture used for producing the mixture of isocyanurates of the invention will now be described in more detail.

### Dicarboxylic Acid

Constituent (a) used in the production of the mixture of (meth)acrylated isocyanurates of the invention comprises at least one dicarboxylic acid. Constituent (a) used in the production of the mixture of (meth)acrylated isocyanurates of the invention may comprise a mixture of dicarboxylic acids.

The dicarboxylic acid may in particular be saturated or unsaturated, linear, branched or cyclic. The dicarboxylic acid may in particular be selected from: saturated aliphatic dicarboxylic acids, unsaturated aliphatic dicarboxylic acids, saturated cycloaliphatic dicarboxylic acids, unsaturated cycloaliphatic dicarboxylic acids, aromatic dicarboxylic acids, and mixtures thereof.

Examples of saturated aliphatic dicarboxylic acids include in particular adipic acid, sebacic acid, succinic acid, 2-methylsuccinic acid, 2-ethylsuccinic acid, 2,2-dimethylsuccinic acid, 1,11-undecanedioic acid, 1,12-dodecanedioic acid, oxalic acid, malonic acid, 2-methylmalonic acid, 2-ethylmalonic acid, glutaric acid, 3,3-dimethylglutaric acid, 3,3-diethylglutaric acid, pimelic acid, suberic acid, azelaic acid or a C₃₂-C₃₆ dimer fatty acid.

Examples of unsaturated aliphatic dicarboxylic acids include in particular itaconic acid, citraconic acid, mesaconic acid, glutaconic acid, muconic acid, fumaric acid or maleic acid.

Examples of saturated cycloaliphatic dicarboxylic acids include in particular cyclopentane-1,2- or -1,3-dicarboxylic acid, cyclohexane-1,2-, -1,3- or -1,4-dicarboxylic acid, cycloheptane-1,2-dicarboxylic acid, and 1,2-, 1,3- or 1,4-bis(carboxymethyl)cyclohexane.

An example of an unsaturated cycloaliphatic dicarboxylic acid is tetrahydrophthalic acid.

Examples of aromatic dicarboxylic acids are phthalic acid, isophthalic acid, terephthalic acid, and bis(4-carboxyphenyl) methane.

In the context of this description, a “dicarboxylic acid” is understood as meaning both the dicarboxylic acids themselves and derivatives of the dicarboxylic acids. Such a derivative can be converted into the dicarboxylic acid by hydrolysis. Dicarboxylic derivatives include the partially or fully esterified forms of the dicarboxylic acids defined above, in particular the C₁-C₆ alkyl mono- and diesters of the dicarboxylic acids defined above and also the corresponding cyclic anhydrides, corresponding amides, and corresponding acyl halides.

Examples of suitable ester-type dicarboxylic acid derivatives are dimethyl malonate, diethyl malonate, dimethyl adipate, dimethyl glutarate, and dimethyl succinate.

The dicarboxylic acid derivative may in particular be a cyclic anhydride. The cyclic anhydride may be saturated or unsaturated, in particular unsaturated. The cyclic anhydride may be cycloaliphatic or aromatic, in particular aromatic.

Examples of saturated cyclic anhydrides are succinic anhydride and hexahydrophthalic anhydride.

Examples of unsaturated cycloaliphatic anhydrides are maleic anhydride, fumaric anhydride, and tetrahydrophthalic anhydride.

An example of an aromatic anhydride is phthalic anhydride.

Dicarboxylic acid derivatives are advantageously selected from diesters and cyclic anhydrides thereof.

The dicarboxylic acids themselves and derivatives thereof may be used alone or in the form of mixtures comprising a plurality of dicarboxylic acids, a plurality of dicarboxylic acid derivatives, or at least one dicarboxylic acid and at least one dicarboxylic acid derivative.

According to a preferred embodiment, the dicarboxylic acid is a saturated aliphatic dicarboxylic acid, preferably a saturated C₄-C₁₀ aliphatic dicarboxylic acid, more preferably a dicarboxylic acid selected from adipic acid, sebacic acid, succinic acid, and mixtures thereof, even more preferably a mixture of succinic and sebacic acid.

### (Meth)Acrylic Monomer

Constituent (b) used in the production of the mixture of (meth)acrylated isocyanurates of the invention comprises at least one (meth)acrylic monomer selected from acrylic acid, methacrylic acid, the anhydrides thereof or mixtures thereof.

In one embodiment of the invention, the (meth)acrylic monomer consists of a mixture of acrylic acid and methacrylic acid, preferably in a molar ratio of from 5:95 to 95:5, more preferably from 5:95 to 15:85 or from 95:5 to 85:15.

It is also preferable that the molar ratio of constituent (a) to constituent (b) is between 1:10 and 1:25, preferably between 1:12 and 1:22, and more preferably between 1:14 and 1:20.

### Monocarboxylic Acid

Constituent (b) used in the production of the mixture of (meth)acrylated isocyanurates of the invention may comprise, in addition to the (meth)acrylic monomer, at least one other C₆-C₂₄ monocarboxylic acid (i.e. a monocarboxylic acid having from 6 to 24 carbon atoms). In this case, it is preferable that the molar ratio of monocarboxylic acid to (meth)acrylic monomer is between 5:95 and 15:85.

The monocarboxylic acid may in particular be saturated or unsaturated, linear or branched. The monocarboxylic acid may in particular be selected from: saturated monocarboxylic acids, monounsaturated monocarboxylic acids, polyunsaturated monocarboxylic acids, and mixtures thereof.

Examples of saturated monocarboxylic acids include in particular hexanoic, heptanoic, octanoic, isooctanoic, nonanoic, isononanoic (or cekanoic), decanoic, undecanoic, dodecanoic, tridecanoic, tetradecanoic, pentadecanoic, hexadecanoic, heptadecanoic, octadecanoic, 12-hydroxyoctadecanoic, nonadecanoic, eicosanoic, and 14-hydroxyeicosanoic acids, and mixtures thereof. For the purposes of the invention, isooctanoic acid is a branched C₈ monocarboxylic acid (i.e. having 8 carbon atoms), and isononanoic acid is a branched C₉ monocarboxylic acid (i.e. having 9 carbon atoms). A particular example of an isononanoic acid is 3,5,5-trimethylhexanoic acid

Examples of monounsaturated monocarboxylic acids include in particular myristoleic acid, palmitoleic acid, sapienic acid, oleic acid, gadoleic acid, ricinoleic acid (12-hydroxy-9-octadecenoic acid), elaidic acid, trans-vaccenic acid, erucic acid, nervonic acid, brassidic acid, lesquerolic acid (14-hydroxy-11-eicosenoic acid), and mixtures thereof.

Examples of polyunsaturated monocarboxylic acids include in particular omega-3 and omega-6 fatty acids, in particular 7,10,13-hexadecatrienoic acid, 9,12,15-octadecatrienoic acid, 6,9,12,15-octadecatetraenoic acid, 11,14,17-eicosatrienoic acid, 8,11,14,17-eicosatetraenoic acid, 5,8,11,14,17-eicosapentaenoic acid, 6,9,12,15,18-heneicosapentaenoic acid, 7,10,13,16,19-docosapentaenoic acid, 4,7,10,13,16,19-docosahexaenoic acid, 9,12,15,18,21-tetracosapentaenoic acid, 6,9,12,15,18,21-tetracosahexaenoic acid, 9,12-octadecadienoic acid, 6,9,12-octadecatrienoic acid, 11,14-eicosadienoic acid, 8,11,14-eicosatrienoic acid, 5,8,11,14-eicosatetraenoic acid, 13,16-docosadienoic acid, 7,10,13,16-docosatetraenoic acid, 4,7,10,13,16-docosapentaenoic acid, 9,12,15,18-tetracosatetraenoic acid, 6,9,12,15,18-tetracosapentaenoic acid, and mixtures thereof.

Monocarboxylic acids may in particular be obtained from vegetable oils.

According to a preferred embodiment, the monocarboxylic acid is isononanoic acid.

### Tris(Hydroxyalkyl)Isocyanurate-THAIC

Constituent (c) used in the production of the mixture of (meth)acrylated isocyanurates of the invention comprises at least one tris(hydroxyalkyl) isocyanurate (THAIC).

The THAIC may in particular be selected from tris(2-hydroxymethyl) isocyanurate, tris(2-hydroxyethyl) isocyanurate, tris(2-hydroxypropyl) isocyanurate, tris(2-hydroxyisopropyl) isocyanurate, tris(3-hydroxypropyl) isocyanurate, tris(2-hydroxybutyl) isocyanurate, tris(4-hydroxybutyl) isocyanurate, and also alkoxylated (in particular ethoxylated and/or propoxylated) derivatives thereof. The tris(hydroxyalkyl) isocyanurate is preferably tris(2-hydroxyethyl) isocyanurate, or THEIC, corresponding to formula (IV) below:

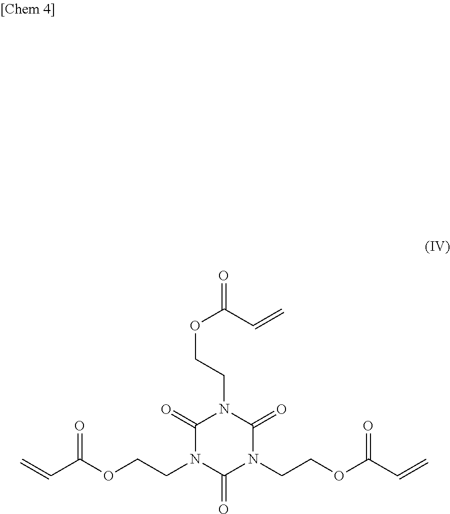 [SMILES: C=CC(=O)OCCn1c(=O)n(CCOC(=O)C=C)c(=O)n(CCOC(=O)C=C)c1=O]

According to a preferred embodiment, THAIC represents from 75 to 100 mol %, preferably 80 to 100 mol %, more preferably 85 to 100 mol %, of the total number of moles of constituent (c).

### Other Polyol P_{OH}

Constituent (c) used in the production of the mixture of (meth)acrylated isocyanurates of the invention may optionally comprise a polyol other than THAIC, also denoted P_{OH}.

Where P_{OH} is present, this may in particular be selected from the following: ethylene glycol, propane-1,2- or -1,3-diol, butane-1,2-, -1,3-2,3- or -1,4-diol, pentane-1,5-diol, hexane-1,6-diol, 3-methylpentane-1,5-diol, decane-1,10-diol, dodecane-1,12-diol, di-, tri- or polyethylene glycol, di-, tri- or polypropylene glycol, cyclohexane-1,4-dimethanol, cyclohexane-1,6-dimethanol, cyclohexane-1,4-diol, bisphenol A, hydrogenated bisphenol A, glycerol, diglycerol, tricyclodecanedimethanol, trimethylolpropane, di(trimethylolpropane), trimethylolethane, hexane-1,2,6-triol, butane-1,2,4-triol, erythritol, pentaerythritol, di(pentaerythritol), neopentyl glycol, 2-butyl-2-ethylpropane-1,3-diol, 2-methylpropane-1,3-diol, 2-methylpropane-1,2-diol, sorbitol, mannitol, xylitol, isosorbide, isoidide, isomannide, methylglucoside, a polyester polyol (in particular polycaprolactone polyol), a polycarbonate polyol, a polyorganosiloxane polyol (in particular polydimethysiloxane polyol), a polyglycerol (in particular polyglycerol-3 (glycerol trimer) and decaglycerol), a hydroxy-terminated polybutadiene, a diol derived from a hydrogenated or non-hydrogenated dimer or trimer fatty acid, alkoxylated (in particular ethoxylated and/or propoxylated) derivatives of the polyols recited above, and mixtures thereof, preferably sorbitol.

According to a preferred embodiment, the reaction mixture, in particular constituent (c), does not contain any polyol other than tris(hydroxyalkyl) isocyanurate.

In all cases, the molar ratio of —COOH groups of constituent (a) to —OH groups of constituent (c) is between 1:4 and 1:20, preferably between 1:5 and 1:15, more preferably between 1:6 and 1:12.

### Process for Preparing Isocyanurate Mixtures

The mixture of (meth)acrylated isocyanurates described above can be obtained by a process comprising the steps of:
1) reacting all or part of a constituent (b) comprising at least one (meth)acrylic monomer selected from acrylic acid, methacrylic acid, anhydrides thereof, and mixtures thereof and optionally at least one C₆-C₂₄ monocarboxylic acid with a constituent (c) comprising at least one tris(hydroxyalkyl) isocyanurate and optionally another polyol P_{OH} under conditions that permit the esterification of from 50 to 95%, preferably from 60 to 90%, more preferably from 70 to 85%, of the OH groups of constituent (c),
2) reacting the mixture from step 1) with a constituent (a) comprising at least one dicarboxylic acid and optionally with any residual amount of constituent (b).

In this process, the totality of the (meth)acrylic monomer may be introduced in step 1) or part of the (meth)acrylic monomer may be introduced in step 1) and the remainder in step 2). In the latter case, it is advantageous when the (meth)acrylic monomers respectively introduced in step 1) and step 2) are different.

Step 1) of the process of the invention is generally carried out in a reactor equipped with a stirring system. It is usually carried out in the presence of an esterification catalyst, a polymerization inhibitor, a solvent, and optionally a dehydrating agent. The esterification reaction is conventionally promoted by removing the water produced during the reaction, in the form of an azeotropic mixture with the solvent. In this step, the reagents may be introduced sequentially or otherwise. The temperature is generally set to between 5° and 120° C. and more preferably to between 8° and 110° C., and the reaction may optionally be carried out under pressure or under reduced pressure.

Examples of solvents that may be used in step 1) are organic hydrocarbon solvents, such as n-hexane, n-heptane, cyclohexane, methylcyclohexane, benzene, toluene or xylene; halogenated organic solvents, such as dichloromethane or trichloroethane; and mixtures thereof. The solvent is preferably an organic hydrocarbon solvent. It may represent from 5% to 150% by weight, and preferably from 50% to 100% by weight, relative to the total amount of (meth)acrylic monomer and polyol.

In turn, the esterification catalyst may be selected in particular from inorganic acids, such as hydrochloric acid, sulfuric acid, and phosphoric acid; salts of inorganic acids, such as diammonium, disodium or dipotassium bisulfate, ammonium, sodium or potassium hydrogen phosphate, ammonium, sodium or potassium phosphate; organic acids, in particular alkyl- or arylsulfonic acids such as para-toluenesulfonic acid, 2-naphthalenesulfonic acid, methanesulfonic acid, trifluoromethanesulfonic acid, ethanesulfonic acid, and benzenesulfonic acid; and mixtures thereof. The catalyst is preferably selected from organic acids. It may represent from 1% to 5%, and preferably from 1.5% to 3.5%, by weight of the total amount of (meth)acrylic monomer and polyol.

Examples of polymerization inhibitors are: quinones, such as hydroquinone, methoxyhydroquinone, para-benzoquinone; catechols, such as tert-butylcatechol; para-hydroxyanisole; mono-, di- and trialkylphenols, such as 2-tert-butyl-4-methylphenol, 6-tert-butyl-2,4-dimethylphenol, 2,6-di-tert-butyl-4-methylphenol, 2-tert-butylphenol, 4-tert-butylphenol 2,4-di-tert-butylphenol, 2-methyl-4-tert-butylphenol, 4-tert-butyl-2,6-dimethylphenol; phenothiazine; phosphorous and hypophosphorous acids; copper or manganese salts, such as copper chloride, copper acetate, copper sulfate, manganese chloride, manganese acetate, and manganese sulfate; and mixtures thereof. The polymerization inhibitor may represent from 0.1% to 2.5%, and preferably from 0.5% to 1.5%, by weight of the total amount of (meth)acrylic monomer and polyol.

The esterification reaction of step 1) is performed under conditions that make it possible to esterify from 50 to 95%, preferably from 60 to 90%, more preferably from 70 to 85%, of the hydroxyl groups of constituent (c), as measured by the acid value, as shown in the examples below.

The mixture obtained at the end of step 1) of the process of the invention comprises (meth)acrylated THAIC, as defined above. The (meth)acrylated THAIC thus comprises a mixture of THAIC mono-, di- and/or tri(meth)acrylates, in which THAIC tri(meth)acrylate (also denoted THAICT(M)A) predominates.

If constituent c) used in step 1) of the process of the invention comprises another polyol P_{OH}, the product obtained at the end of step 1) will also comprise (meth)acrylated P_{OH}, namely a mixture of fully (meth)acrylated Por (resulting from the total esterification of P_{OH} by the (meth)acrylic monomer) and sub (meth)acrylated Por (resulting from the partial esterification of P_{OH} by the (meth)acrylic monomer).

In step 2) of the process of the invention, the dicarboxylic acid and optionally the monocarboxylic acid are reacted with the product of step 1) in order to esterify the residual hydroxyl functions of the (meth)acrylated THAIC and optionally of the (meth)acrylated P_{OH}. This polyesterification step is generally performed under reflux.

The product thus obtained can be isolated by distilling off the water. It is then advantageously washed with an aqueous alkali solution, after which the organic phase is separated, in particular by decantation. The latter can then optionally be subjected to further washing with an aqueous alkali solution or water. Finally, the solvent is distilled off, generally under reduced pressure.

The mixture obtained with the process of the invention may in particular comprise:
THAICT(M)A;
optionally fully (meth)acrylated P_{OH}; and
a polyester component PE.

The polyester component PE may in particular comprise polyesters based on dicarboxylic acid and on mono- and/or di(meth)acrylates of THAIC and optionally on sub (meth)acrylated P_{OH}. The polyester component PE may in particular comprise or consist of a mixture of compounds corresponding to formula (V) below:

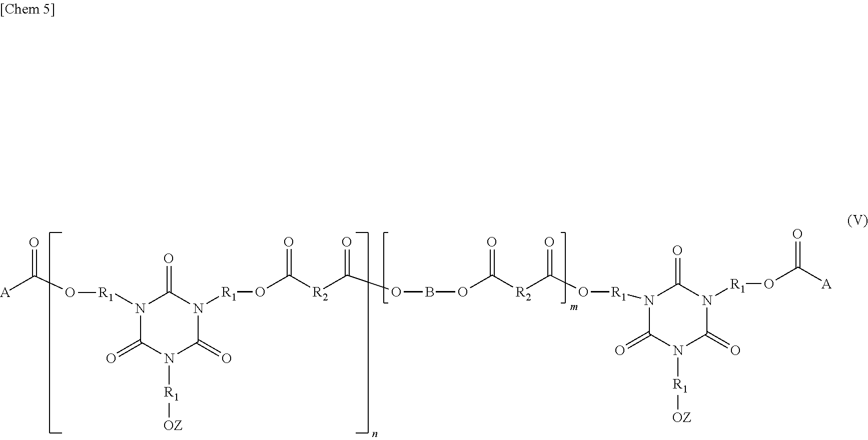

in which:
each R₁ is independently an optionally alkoxylated C₂-C₁₂ alkylene;
each R₂ is independently a dicarboxylic acid residue;
each A is independently a (meth)acrylic acid residue or C₆-C₂₄ monocarboxylic acid residue, preferably a (meth)acrylic acid residue;
each B is independently a polyol P_{OH} residue other than a THAIC residue;
each Z is independently H or —C(═O)-A;
m and n are average values where n ranges from 1 to 10, preferably n ranges from 1 to 2, and m ranges from 0 to 10; preferably, m is equal to 0.

The polyester component PE preferably comprises at least one compound of formula (V) in which at least one of the groups A, preferably each group A, corresponds to a (meth)acrylic acid residue, i.e. a group of formula —C(R₃)═CH₂ in which R₃ is H or methyl. The polyester component PE may in particular comprise at least one compound of formula (V) in which at least one of the groups Z corresponds to a —C(═O)—C(R₃)═CH₂ group. The polyester component PE may in particular comprise at least one compound of formula (V) in which at least one of the groups Z corresponds to a group H.

More preferably, the polyester component PE comprises or consists of a mixture of compounds corresponding to formula (VI) below:

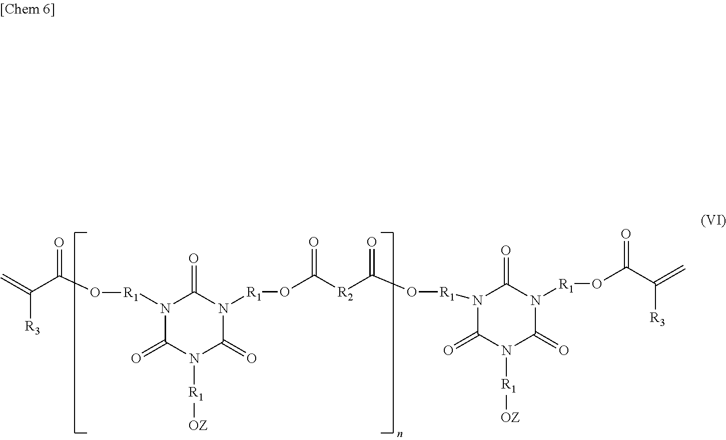

in which:
each R₁ is independently an optionally alkoxylated C₂-C₁₂ alkylene;
each R₂ is independently a dicarboxylic acid residue;
each R₃ is independently H or methyl;
each Z is independently H or —C(═O)—C(R₃)═CH₂;
n ranges from 1 to 10; preferably, n ranges from 1 to 2.

The polyester component PE may in particular comprise at least one compound of formula (VI) in which at least one of the groups Z corresponds to a —C(═O)—C(R₃)═CH₂ group. The polyester component PE may in particular comprise at least one compound of formula (VI) in which at least one of the groups Z corresponds to a group H.

The mixture of the invention may in particular comprise 40% to 90%, preferably 45% to 85%, more preferably 50% to 80%, by weight of THAICT(M)A relative to the weight of the mixture (excluding any solvent).

The mixture of the invention may in particular comprise 10% to 60%, preferably 15% to 55%, more preferably 20% to 50%, by weight of polyester component PE relative to the weight of the mixture (excluding any solvent).

### Polymerizable Composition

The present invention also provides a polymerizable composition comprising at least a mixture of (meth)acrylated isocyanurates as defined according to the present invention and optionally at least one other ethylenically unsaturated compound.

For the purposes of the invention, an “ethylenically unsaturated compound” means a compound that includes a polymerizable carbon-carbon double bond. A polymerizable carbon-carbon double bond is a carbon-carbon double bond that is able to react with another carbon-carbon double bond in a polymerization reaction. A polymerizable carbon-carbon double bond is generally one within a group selected from acrylate (including cyanoacrylate), methacrylate, acrylamide, methacrylamide, styrene, maleate, fumarate, itaconate, allyl, propenyl, vinyl, and corresponding combinations, preferably selected from acrylate, methacrylate, and vinyl, more preferably selected from acrylate and methacrylate. Carbon-carbon double bonds in a phenyl ring are not considered to be polymerizable carbon-carbon double bonds.

In one embodiment, the ethylenically unsaturated compound may be selected from a (meth)acrylate-functionalized monomer, a (meth)acrylate-functionalized oligomer, and corresponding mixtures. In particular, the ethylenically unsaturated compound comprises a (meth)acrylate-functionalized monomer.

The total amount of ethylenically unsaturated compound in the polymerizable composition may be from 0% to 90%, in particular 5% to 85%, more particularly 10% to 80%, by weight based on the total weight of the composition. In particular, the polymerizable composition may comprise 0% to 60%, or 5% to 60% or 10% to 60% or 15% to 60% or 20% to 60%, by weight of ethylenically unsaturated compound based on the weight of the composition. As a variant, the polymerizable composition may comprise 50-80%, or 55-80% or 60-80%, by weight of ethylenically unsaturated compound based on the weight of the composition.

As used herein, the term “(meth)acrylate-functionalized monomer” means a monomer comprising at least one (meth)acryloyloxy group, in particular an acryloyloxy group. The term “(meth)acrylate-functionalized oligomer” means an oligomer comprising a (meth)acryloyloxy group, in particular an acryloyloxy group.

In one embodiment, the ethylenically unsaturated compound comprises a (meth)acrylate-functionalized monomer. The ethylenically unsaturated compound may comprise a mixture of (meth)acrylate-functionalized monomers.

The (meth)acrylate-functionalized monomer may have a molecular weight of less than 600 g/mol, in particular from 100 to 550 g/mol, more particularly from 200 to 500 g/mol.

The (meth)acrylate-functionalized monomer may have 1 to 6 (meth)acryloyloxy groups, in particular 1 to 4 (meth)acryloyloxy groups.

The (meth)acrylate-functionalized monomer may comprise a mixture of (meth)acrylate-functionalized monomers having different functionalities. For example, the (meth)acrylate-functionalized monomer may comprise a mixture of a (meth)acrylate-functionalized monomer containing a single acryloyloxy or methacryloyloxy group per molecule (referred to herein as “mono(meth)acrylate-functionalized compounds”) and a (meth)acrylate-functionalized monomer containing 2 or more, preferably 2 or 3, acryloyloxy and/or methacryloyloxy groups per molecule.

In one embodiment, the (meth)acrylate-functionalized monomer comprises a mono(meth)acrylate-functionalized monomer. The mono(meth)acrylate-functionalized monomer may advantageously function as a reactive diluent and reduce the viscosity of the polymerizable composition of the invention.

Examples of suitable mono(meth)acrylate-functionalized monomers include, but are not limited to, mono(meth)acrylate esters of aliphatic alcohols (the aliphatic alcohol may be straight-chain, branched or alicyclic and may be a monool, diol or polyol, provided just one hydroxyl group has been esterified with (meth)acrylic acid); mono(meth)acrylate esters of aromatic alcohols (such as phenols and including alkylated phenols); mono(meth)acrylate esters of alkylaryl alcohols (such as benzyl alcohol); mono(meth)acrylate esters of oligomeric and polymeric glycols (such as diethylene glycol, triethylene glycol, dipropylene glycol, tripropylene glycol, polyethylene glycol and polypropylene glycol); mono(meth)acrylate esters of monoalkyl ethers of glycols and oligoglycols; mono(meth)acrylate esters of alkoxylated (for example ethoxylated and/or propoxylated) aliphatic alcohols (the aliphatic alcohol may be straight-chain, branched or alicyclic and may be a monool, diol or polyol, provided just one hydroxyl group of the alkoxylated aliphatic alcohol has been esterified with (meth)acrylic acid); mono(meth)acrylate esters of alkoxylated (for example ethoxylated and/or propoxylated) aromatic alcohols (such as alkoxylated phenols); caprolactone mono(meth)acrylates; and the like.

The following compounds are specific examples of mono(meth)acrylate-functionalized monomers suitable for use in the polymerizable compositions of the present invention: methyl (meth)acrylate; ethyl (meth)acrylate; n-propyl (meth)acrylate; n-butyl (meth)acrylate; isobutyl (meth)acrylate; n-hexyl (meth)acrylate; 2-ethylhexyl (meth)acrylate; n-octyl (meth)acrylate; isooctyl (meth)acrylate; n-decyl (meth)acrylate; n-dodecyl (meth)acrylate; tridecyl (meth)acrylate; tetradecyl (meth)acrylate; hexadecyl (meth)acrylate; 2-hydroxyethyl (meth)acrylate; 2-hydroxypropyl (meth)acrylate and 3-hydroxypropyl (meth)acrylate; 2-methoxyethyl (meth)acrylate; 2-ethoxyethyl (meth)acrylate; 2-ethoxypropyl (meth)acrylate and 3-ethoxypropyl (meth)acrylate; tetrahydrofurfuryl (meth)acrylate; alkoxylated tetrahydrofurfuryl (meth)acrylate; 2-(2-ethoxyethoxy) ethyl (meth)acrylate; cyclohexyl (meth)acrylate, glycidyl (meth)acrylate; isodecyl (meth)acrylate; lauryl (meth)acrylate; 2-phenoxyethyl (meth)acrylate; alkoxylated phenol (meth)acrylates; alkoxylated nonylphenol (meth)acrylates; cyclic trimethylolpropane formal (meth)acrylate; isobornyl (meth)acrylate; tricyclodecanemethanol (meth)acrylate; tert-butylcyclohexanol (meth)acrylate; trimethylcyclohexanol (meth)acrylate; diethylene glycol monomethyl ether (meth)acrylate; diethylene glycol monobutyl ether (meth)acrylate; triethylene glycol monoethyl ether (meth)acrylate; ethoxylated lauryl (meth)acrylate; methoxypolyethylene glycol (meth)acrylates; hydroxyl ethyl-butyl urethane (meth)acrylates; 3-(2-hydroxyalkyl) oxazolidinone (meth)acrylates; and combinations thereof.

In one embodiment, the (meth)acrylate-functionalized monomer may comprise a (meth)acrylate-functionalized monomer containing two or more (meth)acryloyloxy groups per molecule.

Examples of suitable (meth)acrylate-functionalized monomers containing two or more (meth)acryloyloxy-type groups per molecule include acrylate and methacrylate esters of polyols (organic compounds containing two or more hydroxyl groups per molecule, for example 2 to 6). Specific examples of suitable polyols are as defined previously for P and P′. Such polyols may be totally or partially esterified (with a (meth)acrylic acid, a (meth)acrylic anhydride, a (meth)acryloyl chloride or the like), provided they contain at least two (meth)acryloyloxy-type functional groups per molecule.

Examples of functionalized (meth)acrylate monomers containing two or more (meth)acryloyloxy groups per molecule may include bisphenol A di(meth)acrylate; hydrogenated bisphenol A di(meth)acrylate; ethylene glycol di(meth)acrylate; diethylene glycol di(meth)acrylate; triethylene glycol di(meth)acrylate; tetraethylene glycol di(meth)acrylate; polyethylene glycol di(meth)acrylate; propylene glycol di(meth)acrylate; dipropylene glycol di(meth)acrylate; tripropylene glycol di(meth)acrylate; tetrapropylene glycol di(meth)acrylate; polypropylene glycol di(meth)acrylate; polytetramethylene glycol di(meth)acrylate; butane-1,2-diol di(meth)acrylate; butane-2,3-diol di(meth)acrylate; butane-1,3-diol di(meth)acrylate; butane-1,4-diol di(meth)acrylate; pentane-1,5-diol di(meth)acrylate; hexane-1,6-diol di(meth)acrylate; octane-1,8-diol di(meth)acrylate; nonane-1,9-diol di(meth)acrylate; decane-1,10-diol di(meth)acrylate; dodecane-1,12-diol di(meth)acrylate; neopentyl glycol di(meth)acrylate; 2-methylpentane-2,4-diol di(meth)acrylate; polybutadiene di(meth)acrylate; cyclohexane-1,4-dimethanol di(meth)acrylate; tricyclodecanedimethanol di(meth)acrylate; metal di(meth)acrylates; modified metal di(meth)acrylates; glycerol di(meth)acrylate; glycerol tri(meth)acrylate; trimethylolethane tri(meth)acrylate; trimethylolethane di(meth)acrylate; trimethylolpropane tri(meth)acrylate; trimethylolpropane di(meth)acrylate; pentaerythritol di(meth)acrylate; pentaerythritol tri(meth)acrylate; pentaerythritol tetra(meth)acrylate, di(trimethylolpropane) di(meth)acrylate; di(trimethylolpropane) tri(meth)acrylate; di(trimethylolpropane) tetra(meth)acrylate; sorbitol penta(meth)acrylate; di(pentaerythritol) tetra(meth)acrylate; di(pentaerythritol) penta(meth)acrylate; di(pentaerythritol) hexa(meth)acrylate; tris(2-hydroxyethyl) isocyanurate tri(meth)acrylate; and also alkoxylated (e.g. ethoxylated and/or propoxylated) derivatives thereof; and mixtures thereof.

The polymerizable composition of the invention may comprise 0% to 90%, in particular 5% to 85%, more particularly 10% to 80%, by weight of (meth)acrylate-functionalized monomer based on the weight of the composition. In particular, the polymerizable composition may comprise 0% to 60%, or 5% to 60%, or 10% to 60%, or 15% to 60%, or 20% to 60%, by weight of (meth)acrylate . . . functionalized monomer based on the weight of the composition. As a variant, the polymerizable composition may comprise 50% to 80%, or 55% to 80%, or 60% to 80%, by weight of (meth)acrylate-functionalized monomer based on the weight of the composition.

In one embodiment, the ethylenically unsaturated compound comprises a (meth)acrylate-functionalized oligomer. The ethylenically unsaturated compound may comprise a mixture of (meth)acrylate-functionalized oligomers.

The (meth)acrylate-functionalized oligomer may be chosen so as to enhance the flexibility, strength and/or modulus, among other attributes, of a cured polymer prepared using the polymerizable composition of the present invention.

The (meth)acrylate-functionalized oligomer may have 1 to 18 (meth)acryloyloxy groups, in particular 2 to 6 (meth)acryloyloxy groups, more particularly 2 to 6 acryloyloxy groups.

The (meth)acrylate-functionalized oligomer may have a number-average molecular weight of greater than or equal to 600 g/mol, in particular 800 to 15 000 g/mol, more particularly 1000 to 5000 g/mol. In particular, the (meth)acrylate-functionalized oligomers may be selected from the group consisting of (meth)acrylate-functionalized urethane oligomers (sometimes also referred to as “urethane (meth)acrylate oligomers”, “polyurethane (meth)acrylate oligomers” or “carbamate (meth)acrylate oligomers”), (meth)acrylate-functionalized epoxy oligomers (sometimes also referred to as “epoxy (meth)acrylate oligomers”), (meth)acrylate-functionalized polyether oligomers (sometimes also referred to as “polyether (meth)acrylate oligomers”), (meth)acrylate-functionalized polydiene oligomers (sometimes also referred to as “polydiene (meth)acrylate oligomers”), (meth)acrylate-functionalized polycarbonate oligomers (sometimes also referred to as “polycarbonate (meth)acrylate oligomers”), and (meth)acrylate-functionalized polyester oligomers (sometimes also referred to as “polyester (meth)acrylate oligomers”) other than that of the invention and corresponding mixtures.

Polyester (meth)acrylate oligomers include, for example, products of the reaction of acrylic or methacrylic acid or corresponding synthetic mixtures or equivalents with hydroxy-terminated polyester polyols. The reaction process can be performed so that all, or essentially all, the hydroxyl groups of the polyester polyol have been (meth)acrylated, particularly where the polyester polyol is difunctional. Polyester polyols may be prepared by polycondensation reactions of polyhydroxy-functionalized components (in particular of diols) and poly(carboxylic acid)-functionalized compounds (in particular of dicarboxylic acids and anhydrides). The polyhydroxy-functionalized and poly(carboxylic acid)-functionalized components may each have linear, branched, cycloaliphatic or aromatic structures and may be used individually or as mixtures.

Examples of suitable epoxy (meth)acrylates include products of the reaction of acrylic or methacrylic acid or corresponding mixtures with an epoxy resin (polyglycidyl ether or ester). The epoxy resin may in particular be selected from bisphenol A diglycidyl ether, bisphenol F diglycidyl ether, bisphenol S diglycidyl ether, brominated bisphenol A diglycidyl ether, brominated bisphenol F diglycidyl ether, brominated bisphenol S diglycidyl ether, epoxy novolac resin, hydrogenated bisphenol A diglycidyl ether, hydrogenated bisphenol F diglycidyl ether, hydrogenated bisphenol S diglycidyl ether, 3,4-epoxycyclohexylmethyl 3′,4′-epoxycyclohexanecarboxylate, 2-(3,4-epoxycyclohexyl-5,5-spiro-3,4-epoxy) cyclohexane-1,4-dioxane, bis(3,4-epoxycyclohexylmethyl) adipate, vinylcyclohexene oxide, 4-vinylepoxycyclohexane, bis(3,4-epoxy-6-methylcyclohexylmethyl) adipate, 3,4-epoxy-6-methylcyclohexyl 3′,4′-epoxy-6′-methylcyclohexanecarboxylate, methylenebis(3,4-epoxycyclohexane), dicyclopentadiene diepoxide, ethylene glycol di(3,4-epoxycyclohexylmethyl) ether, ethylenebis(3,4-epoxycyclohexanecarboxylate), butane-1,4-diol diglycidyl ether, hexane-1,6-diol diglycidyl ether, glycerol triglycidyl ether, trimethylolpropane triglycidyl ether, polyethylene glycol diglycidyl ether, polypropylene glycol diglycidyl ether, polyglycidyl ethers of a polyether polyol obtained by the addition of one or more alkylene oxides to an aliphatic polyhydric alcohol, such as ethylene glycol, propylene glycol, and glycerol, diglycidyl esters of long-chain dibasic aliphatic acids, monoglycidyl ethers of aliphatic higher alcohols, monoglycidyl ethers of phenol, cresol, butylphenol, or polyether alcohols obtained by the addition of alkylene oxide to these compounds, glycidyl esters of higher fatty acids, epoxidized soybean oil, epoxybutylstearic acid, epoxyoctylstearic acid, epoxidized linseed oil, an epoxidized polybutadiene, and the like.

Suitable polyether (meth)acrylate oligomers include, but are not limited to, condensation reaction products of acrylic or methacrylic acid or of corresponding mixtures or synthetic equivalents with polyetherols that are polyether polyols (such as a polyethylene glycol, a polypropylene glycol or a polytetramethylene glycol). Suitable polyetherols may be linear or branched substances containing ether linkages and terminal hydroxyl groups. The polyetherols may be prepared by ring-opening polymerization of cyclic ethers such as tetrahydrofuran or alkylene oxides (for example ethylene oxide and/or propylene oxide) with a starter molecule. Suitable starter molecules include water, polyhydroxy-functionalized materials, polyester polyols, and amines.

Polyurethane (meth)acrylate oligomers (sometimes also referred to as “urethane (meth)acrylate oligomers”) suitable for use in the polymerizable compositions of the present invention include urethanes based on aliphatic, cycloaliphatic and/or aromatic polyester polyols and polyether polyols and aliphatic, cycloaliphatic and/or aromatic polyester diisocyanates and polyether diisocyanates capped with terminal (meth)acrylate groups. Suitable polyurethane (meth)acrylate oligomers include, for example, aliphatic polyester-based urethane diacrylate and tetraacrylate oligomers, aliphatic polyether-based urethane diacrylate and tetraacrylate oligomers, and also aliphatic polyester/polyether-based urethane diacrylate and tetraacrylate oligomers.

Polyurethane (meth)acrylate oligomers may be prepared by reacting aliphatic, cycloaliphatic or aromatic polyisocyanates (for example diisocyanate, triisocyanate) with polyester polyols, polyether polyols, polycarbonate polyols, polycaprolactone polyols, polyorganosiloxane polyols (for example polydimethylsiloxane polyols), or polydiene polyols (for example polybutadiene polyols), terminated with an OH group, or corresponding combinations, to form isocyanate-functionalized oligomers, which are then reacted with hydroxy-functionalized (meth)acrylates such as hydroxyethyl acrylate or hydroxyethyl methacrylate to provide terminal (meth)acrylate groups. For example, polyurethane (meth)acrylate oligomers may contain two, three, four or more (meth)acrylate functional groups per molecule. Different orders of addition may also be employed for the preparation of the polyurethane (meth)acrylate, as is known in the prior art. For example, the hydroxy-functionalized (meth)acrylate may first be reacted with a polyisocyanate to obtain an isocyanate-functionalized (meth)acrylate, which may then be reacted with an OH-terminated polyester polyol, polyether polyol, polycarbonate polyol, polycaprolactone polyol, polydimethylsiloxane polyol or polybutadiene polyol, or a corresponding combination. In yet another embodiment, a polyisocyanate may first be reacted with a polyol, including any one of the previously mentioned polyol types, to obtain an isocyanate-functionalized polyol, which is then reacted with a hydroxy-functionalized (meth)acrylate to give a polyurethane (meth)acrylate. As a variant, all the components may be combined and reacted at the same time.

The polymerizable composition of the invention may comprise 0% to 90%, in particular 5% to 85%, more particularly 10% to 80%, by weight of (meth)acrylate-functionalized oligomer based on the weight of the composition. In particular, the polymerizable composition may comprise 0 to 60%, or 5% to 60%, or 10% to 60%, or 15% to 60%, or 20% to 60%, by weight of (meth)acrylate-functionalized oligomer based on the weight of the composition. As a variant, the polymerizable composition may comprise 50% to 80%, or 55% to 80%, or 60% to 80%, by weight of (meth)acrylate-functionalized oligomer based on the weight of the composition.

The polymerizable composition of the invention may also advantageously comprise a free-radical or ionic polymerization initiator, and more particularly a photoinitiator or a peroxide.

The photoinitiator may be a free-radical photoinitiator, in particular a free-radical photoinitiator having Norrish I-type activity and/or Norrish II-type activity, more particularly a free-radical photoinitiator having Norrish I-type activity.

Non-limiting types of free-radical photoinitiators suitable for use in the polymerizable compositions of the present invention include, for example, benzoins, benzoin ethers, acetophenones, α-hydroxyacetophenones, benzil, benzil ketals, anthraquinones, phosphine oxides, acylphosphine oxides, α-hydroxyketones, phenylglyoxylates, α-aminoketones, benzophenones, thioxanthones, xanthones, acridine derivatives, phenazene derivatives, quinoxaline derivatives, triazine compounds, benzoyl formates, aromatic oximes, metallocenes, acylsilyl or acylgermanyl compounds, camphorquinones, corresponding polymer derivatives, and corresponding mixtures.

Examples of suitable free-radical photoinitiators include, but are not limited to, 2-methylanthraquinone, 2-ethylanthraquinone, 2-chloroanthraquinone, 2-benzylanthraquinone, 2-t-butylanthraquinone, 1,2-benzo-9,10-anthraquinone, benzil, benzoins, benzoin ethers, benzoin, benzoin methyl ether, benzoin ethyl ether, benzoin isopropyl ether, α-methylbenzoin, α-phenylbenzoin, Michler's ketone, acetophenones such as 2,2-dialkoxybenzophenones and 1-hydroxyphenyl ketones, benzophenone, 4,4′-bis(diethylamino)benzophenone, acetophenone, 2,2-diethyloxyacetophenone, diethyloxyacetophenone, 2-isopropylthioxanthone, thioxanthone, diethylthioxanthone, 1,5-acetonaphthylene, benzil ketone, α-hydroxy keto, 2,4,6-trimethylbenzoyldiphenylphosphine oxide, benzil dimethyl ketal, 2,2-dimethoxy-1,2-diphenylethanone, 1-hydroxycyclohexyl phenyl ketone, 2-methyl-1-[4-(methylthio)phenyl]-2-morpholinopropanone-1,2-hydroxy-2-methyl-1-phenylpropanone, an oligomeric α-hydroxy ketone, benzoylphosphine oxides, phenylbis(2,4,6-trimethylbenzoyl) phosphine oxide, ethyl(2,4,6-trimethylbenzoyl) phenyl phosphinate, anisoin, anthraquinone, anthraquinone-2-sulfonic acid sodium salt monohydrate, (benzene) tricarbonylchromium, benzil, benzoin isobutyl ether, benzophenone/1-hydroxycyclohexyl phenyl ketone 50/50 mixture, 3,3′,4,4′-benzophenonetetracarboxylic dianhydride, 4-benzoylbiphenyl, 2-benzyl-2-(dimethylamino)-4′-morpholinobutyrophenone, 4,4′-bis(diethylamino)benzophenone, 4,4′-bis(dimethylamino)benzophenone, camphorquinone, 2-chlorothioxanthen-9-one, dibenzosuberenone, 4,4′-dihydroxybenzophenone, 2,2-dimethoxy-2-phenylacetophenone, 4-(dimethylamino)benzophenone, 4,4′-dimethylbenzil, 2,5-dimethylbenzophenone, 3,4-dimethylbenzophenone, diphenyl(2,4,6-trimethylbenzoyl) phosphine oxide/2-hydroxy-2-methylpropiophenone 50/50 mixture, 4′-ethoxyacetophenone, 2,4,6-trimethylbenzoyldiphenylphosphine oxide, phenylbis(2,4,6-trimethylbenzoyl) phosphine oxide, ferrocene, 3′-hydroxyacetophenone, 4′-hydroxyacetophenone, 3-hydroxybenzophenone, 4-hydroxybenzophenone, 1-hydroxycyclohexyl phenyl ketone, 2-hydroxy-2-methylpropiophenone, 2-methylbenzophenone, 3-methylbenzophenone, methylbenzoyl formate, 2-methyl-4′-(methylthio)-2-morpholinopropiophenone, phenanthrenequinone, 4′-phenoxyacetophenone, (cumene) cyclopentadienyliron (II) hexafluorophosphate, 9,10-diethoxyanthracene and 9,10-dibutoxyanthracene, 2-ethyl-9,10-dimethoxyanthracene, thioxanthen-9-one, and corresponding combinations.

In particular, the photoinitiator may be a benzophenone (such as SpeedCure® BP, SpeedCure® 7005, SpeedCure® 7006), a thioxanthone (such as SpeedCure® 7010, SpeedCure® ITX), an α-hydroxyacetophenone (such as SpeedCure® 73), an acylphosphine oxide (such as SpeedCure® BPO, SpeedCure® TPO, SpeedCure® TPO-L). Preferably, the photoinitiator is an α-hydroxyacetophenone or an acylphosphine oxide.

The polymerizable composition of the invention may especially comprise 0% to 20%, in particular 0.1% to 15%, more particularly 1% to 10%, by weight of photoinitiator relative to the weight of the composition.

In addition, the polymerizable composition of the invention may comprise one or more additives selected from: antioxidants, photostabilizers, light absorbers, polymerization inhibitors, antifoams, antistatic agents, leveling agents, dispersants (wetting agents, surfactants), slip agents, adhesion promoters, lubricants, pigments, dyes, fillers, chain-transfer agents, rheological agents (thixotropic agents, thickeners), matting agents, opacifiers, impact-resistance agents, and waxes.

Preferably, the polymerizable composition of the invention is a composition for inks, coatings (especially protective coatings, electrical insulation coatings, decorative coatings or coatings reactive to external stimuli), materials filled with fibrous or particulate reinforcements which may be carbon nanotubes or graphite (especially putty, chemical dowel, artificial stone, dental material or composite), an adhesive composition, molding composition, ink plate composition or electrode binder composition, or a composition for additive manufacturing, in particular for the 3D or 4D printing of articles.

For the purposes of the invention, an ink plate is a flexible photopolymer plate intended for transferring ink to the support to be printed in rotary letterpress printing or flexography.

Additive manufacturing, also termed 3D printing, consists of creating a (volumetric/three-dimensional) article point-by-point (these are termed voxels by analogy with pixels in conventional two-dimensional printing) from a digital model containing the properties associated with the geometry of the article to be produced (mesh of points or surfaces) and optionally the parameters of the materials to be used, or by selectively modifying the properties of a soft medium at these points, for example by solidification (polymerization) from a vat of liquid resin, or by agglomeration/sintering/melting-resolidification from a bed of powder, or by selectively depositing the material at different points on a surface (also known as a layer, and generally flat), either continuously (by extrusion) or discontinuously (by inkjet), and doing so surface by surface. The surfaces can be added one under the other or one on top of the other, and also from the center outward, generally starting from a printing support, the unmodified material itself possibly being the support. The general principles of 3D printing are defined in the standard ISO/ASTM 52900:2015. Printing a 4D article may be defined as printing a 3D article that is capable of being transformed over time. Thus, 4D printing is the process by which a 3D-printed article can modify its own structure and change form under the impulse of external energy such as temperature, light or other environmental stimuli.

The polymerizable composition defined above may be crosslinked, in particular by exposing said composition to radiation, and more particularly to UV, near-UV, visible, infrared or near-infrared radiation, or to an electron beam, so as to obtain a crosslinked product that is advantageously an ink, a coating (especially a protective coating, an electrical insulation coating, a decorative coating or a coating that is reactive to external stimuli), a material filled with fibrous or particulate reinforcements that may be carbon nanotubes or graphite (especially a putty, a chemical dowel, an artificial stone, a dental material or a composite), an adhesive, a molded material, an ink plate, an electrode binder, or an article obtained by additive manufacturing, in particular an article obtained by 3D or 4D printing.

Alternatively, it can be used in a process for manufacturing a three-dimensional article comprising an additive manufacturing step, in particular a continuous or layer-by-layer printing step.

The invention is also directed to the use of a mixture of (meth)acrylated isocyanurates of the present invention as a binder in a polymerizable composition.

Finally, the invention lastly provides for the use of a mixture of (meth)acrylated isocyanurates of the present invention in a composition for additive manufacturing, in particular for the 3D or 4D printing of an article.

### EXAMPLES

A better understanding of the invention will be obtained in the light of the examples that follow, which are given purely by way of illustration and are not intended to limit the scope of the invention as defined by the appended claims.

### Starting Materials

The following starting materials were used in the examples:

TABLE 1

| Abbreviation | Chemical name | Function | Supplier |
|---|---|---|---|
| THEIC | Tris(2-hydroxyethyl) | Component c) | Jining |
| | isocyanurate | | Jianbang |
| NPG | Neopentyl glycol | Component c) | Perstorp |
| AA | Acrylic acid | Component b) | Arkema |
| Succ | Succinic acid | Component a) | Roquette |
| Ad | Adipic acid | Component a) | Solvay |
| Seb | Sebacic acid | Component a) | Arkema |
| Cek | Cekanoic acid | Component b) | Brenntag |
| | (isononanoic acid) | | |
| HQ | Hydroquinone | Inhibitor | Solvay |
| PTZ | Phenothiazine | Inhibitor | IMCD |
| Tol | Toluene | Solvent | Total |
| Hept | N-Heptane | Solvent | Total |
| MSA | Methanesulfonic acid | Catalyst | Arkema |
| | (70% by weight | | |
| | aqueous solution) | | |
| SpeedCure 73 | 2-Hydroxy-2-methyl-1- | Photoinitiators | Arkema |
| | phenylpropanone | | |

### Methods

The following methods were used in the present application:

### Content of THAICT(M)A (or THEICT(M)A)

The content of THAICT(M)A (or THEICT (M)A) is calculated from the probability that a (meth)acrylation will occur three times on the same molecule of THAIC (or THEIC), based on the (meth)acrylic functions available as a fraction of the total acid functions in play.

### (Meth)Acrylate Functionality

The (meth)acrylate functionality is calculated from the average elongation n, the overall structure being represented by the following formula:

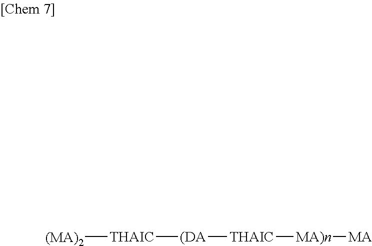

where:
MA: Monoacid residue of component (b) (in particular (meth)acrylic acid and/or cekanoic acid),
DA: Diacid residue of component (a) (in particular succinic acid, adipic acid and/or sebacic acid),
THAIC: THAIC (or THEIC) residue

For n=0, the structure for the THAIC triester represented by the following formula is obtained:

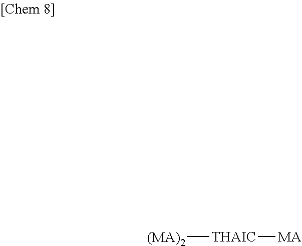

For an assumed total conversion of the hydroxyl functions of THAIC, the average elongation of the polyester is related to the molar ratio of polyacid to polyol=(a)/(c), according to the following equation:

$$( a ) / ( c ) = n / ( n + 1 ) \to n = r / ( 1 - r ) with r = ( a ) / ( c ) \qquad [ Math 1 ]$$

The total functionality f_{TOT}, where this is the number of monoacid termini per average molecule (overall structure), is: f_{TOT}=n+3

The (meth)acrylate functionality f_{ACR} is calculated according to the following equation:

$${f}_{ACR} = {f}_{TOT} \cdot {x}_{ACR} \qquad [ Math 2 ]$$

where x_{ACR}=molar fraction of (meth)acrylic acid in component (b) (corresponding to the ratio of the number of moles of (meth)acrylic acid to the total number of moles of component (b))

The (meth)acrylate functionality f_{ACR} can therefore be calculated according to the following equation:

$${f}_{ACR} = [ ( r / ( 1 - r ) ) + 3 ] \cdot {x}_{A C R} \qquad [ Math 3 ]$$

### Coloration

The APHA (American Public Health Association) color value is defined by a standard range of reference solutions of increasing coloration. According to standard ISO6271, APHA values from 10 to 500 are assigned to solutions of potassium hexachloroplatinate in water of known concentrations, corresponding to an amount in mg of platinum per ml of solution.

### Viscosity

The viscosity is measured according to the Noury method. The time taken for a steel ball to travel under its own gravitational force through the liquid to be characterized is measured. AFNOR XP.T51-213 specifies the geometry of the container, the diameter of the ball (2 mm) and the path of the ball (104 mm). Under these conditions, the dynamic viscosity is proportional to the travel time of the ball, a travel time of 1 second corresponding to a viscosity of 0.1 Pa·s.

### Recrystallization Index

A sample of product deposited on the slide and seeded with a THEICTA crystal is observed (for 6 days and at regular intervals) by optical microscopy. A comparative value (from 0 to 5) is awarded:
no propagation of crystallization: 0
total propagation of crystallization to the whole sample: 5

### Glass Transition Temperature (Tα)

Film preparation:

96% by weight of product to be tested is mixed with 4% by weight of photoinitiator (SpeedCure 73), then this mixture is applied to a glass plate using a 150 μm thickness filmograph.

The films obtained are crosslinked under a Fusion® mercury lamp (UV Hg) (see crosslinking rate test below). The films thus crosslinked are detached from the support and placed between 2 glass plates and annealed by means of 5 passes under the same UV lamp at a speed of 5 m/min.

Dynamic mechanical analysis:

The films are tested by AMD using an RSAII (Rheometrics®) apparatus

Tensile stress, at a frequency of 1 Hz

Temperature ramp: −50° C. to 300° C., at a rate of 3° C./min

### Acid Value (AV)

The acid value of a product is expressed in milligrams of KOH equivalent per gram of product to be characterized. For this, an acid-base titration is carried out under the following conditions: an exact weight m of product (approximately 10 grams) is dissolved in 50 ml of a toluene/ethanol mixture (2:1 vol/vol). Once dissolution is complete, the mixture is titrated with a methanolic potassium hydroxide solution of normality N (eq/L) of approximately 0.1 eq/liter. The equivalence point is detected by a combination electrode controlling an automated burette (716 DMS Titrino® automated titrator from Metrohm) which then delivers an equivalent volume VE. After performing a blank test (50 ml of the toluene/ethanol mixture (2:1 vol/vol) on its own), which makes it possible to determine the equivalent volume VB, the acid value (AV) is calculated by the following equation:

$$A V = [ ( VE - VB ) \cdot N \cdot 56 , 1 ] / m \qquad [ Math 4 ]$$

where VE and VB are expressed in ml, N in eq/liter, and m in grams.

### Reactivity Under a Fusion® Mercury Lamp (UV Hg)

The formulations are applied in a 12 μm film on a “Form 1B Penoparc” contrast card from Leneta®, then crosslinked by irradiating at 120 W/cm² with a Fusion® mercury lamp. The minimum speed of passage under the lamp (in m/min) necessary to obtain a film dry to the touch is measured.

### Flexibility

The formulations are applied in a 100 μm film on a flexible steel plate 25/10 mm thick, then crosslinked by irradiating at 120 W/cm² with a Fusion® mercury lamp at a speed of 10 m/min (2 passes). After 24 hours of post-crosslinking at 23° C., the coated steel plate is bent around cylindrical mandrels. The flexibility is the value (in mm) of the smallest radius of curvature that can be applied to the coating without it cracking or peeling off its support.

### Persoz Hardness

The formulations are applied in a 100 μm film on a glass plate, then crosslinked by irradiating at 120 W/cm² with a Fusion® mercury lamp at a speed of 10 m/min (2 passes). After 24 hours of post-crosslinking at 23° C., the hardness is determined by the number of oscillations before damping (the latter decreasing from 12° to 4° amplitude) of a pendulum in contact with the coated glass plate.

### Acetone Resistance

The formulations are applied in a 12 μm film on a glass plate, then crosslinked by irradiating at 120 W/cm² with a Fusion® mercury lamp at a speed of 10 m/min (2 passes). After 24 hours of post-crosslinking at 23° C., the coating is rubbed with a cloth soaked in acetone. The acetone resistance is the time (in seconds) it takes for the coating to peel off the support and/or to disintegrate.

### Stain Resistance

The formulations are applied in a 12 μm film on a Leneta contrast card, then crosslinked by irradiating at 120 W/cm² with a Fusion® mercury lamp at a speed of 10 m/min (2 passes). After 24 hours of post-crosslinking at 23° C., absorbent paper discs are placed on the card and coffee, perfume (2 ml), and iodine (3 drops) are deposited thereon.

After a contact time of 12 hours, the stains are qualitatively evaluated after removing the discs and cleaning the surface with water:
0: no trace, to 5: very significant mark.

### Example 1: Process for Preparing a Mixture of Isocyanurates of the Invention

A 1-liter reactor equipped with an anchor stirrer, a Dean-Stark apparatus, an air bubbler (flow rate=0.5 liter/hour), and a thermometer is successively charged with the following starting materials: THEIC (component (c): 261.0 g, 1.000 mol), AA (component (b): 202.8 g, 2.817 mol), the solvent (mixture of Hept (82 g) and Tol (328 g), i.e. an overall amount of solvent of 40% by weight based on the total mass charged, this solvent mixture consisting of 80% toluene and 20% heptane), MSA (7.830 g, 3% by weight relative to polyol), HQ (6.084 g, 3% by weight relative to AA), and PTZ (0.008 g, 40 ppm relative to AA). This mixture is heated at 60° C. for one hour until the AV (weak acidity corresponding to the unreacted carboxylic acid groups) reaches a value of less than 60 mg

KOH/g, i.e. about 80% conversion of the carboxylic acid functions.

The Ad (component (a): 24.333 g, 0.167 mol) is then added and the reaction mixture is heated under reflux further until the residual acid value reaches a value of less than 20 mg KOH/g and remains broadly constant (ΔAV in one hour <0.1 mg KOH/g decrease).

At the end of the polyesterification reaction, about 50 ml of water has been distilled off, which corresponds to 95% conversion of the COOH groups. A clear reaction mixture (without turbidity) that is brownish in appearance is recovered. The density is then adjusted to 0.95 g/l by adding the same mixture of toluene and heptane (Tol/Hept ratio by weight=80/20).

This organic phase is neutralized by adding 40 g of NaOH solution (25% by weight aqueous solution). The temperature is about 50° C., the shaking time is 2 minutes, and the settling time is one hour. This organic phase is then washed three times with 30 g of NaOH solution (25% by weight aqueous solution). The temperature is about 50° C., the shaking time is 2 minutes, and the settling time is one hour, with the exception of the final wash, which requires 15 minutes of shaking time. The organic phase is then washed twice with 30 g of water at a temperature of about 55° C., a shaking time of 5 minutes, and a settling time of one hour. The organic phase thus purified is then distilled under reduced pressure (4 hours at 95° C. under a pressure of 0.01 MPa) to remove the solvents (toluene and n-heptane).

### Examples 2-3 and Comparative Examples

The same process as in example 1 described above is retained, but replacing the compounds (a), (b), and (c) as reactants with the amounts (in moles) of the table of examples below. The overall amount of solvent is maintained at 40% by weight of the total load using a toluene+n-heptane mixture (Tol/Hept=80/20 by weight). The MSA catalyst is maintained at a ratio of 3% by weight relative to the polyol. The inhibitors are in turn maintained at a ratio of 3% by weight for HQ and 40 ppm for PTZ, in both cases relative to acrylic acid.

The same procedure applies for the conversion criterion before addition of the polyacid(s) and for the final total conversion. The steps of density adjustment, neutralization, washing, and solvent distillation are identical.

The amounts of reagent for each example are detailed in the table below:

TABLE 2

| | | | | | | | | Example 9 |
|---|---|---|---|---|---|---|---|---|
| | | | | | | | | of |
| | | THEICTA | Cekanoates | | | | | JP94081782 |
| | | Comp. | Comp. | Comp. | THEIC-based polyester | | | Comp. |
| Component | Product | ex. 1 | ex. 2 | ex. 3 | Ex. 1 | Ex. 2 | Ex. 3 | ex. 4 |
| (c) | THEIC | 1.000 | 1.000 | 1.000 | 1.000 | 1.000 | 1.000 | 1.000 |
| | NPG | 0.000 | 0.000 | 0.000 | 0.000 | 0.000 | 0.000 | 1.000 |
| (b) | AA | 3.292 | 2.800 | 3.050 | 2.817 | 2.817 | 2.583 | 2.000 |
| | Cek | 0.000 | 0.500 | 0.250 | 0.000 | 0.000 | 0.250 | 0.000 |
| (a) | Succ | 0.000 | 0.000 | 0.000 | 0.000 | 0.056 | 0.058 | 0.000 |
| | Ad | 0.000 | 0.000 | 0.000 | 0.167 | 0.056 | 0.00 | 1.000 |
| | Seb | 0.000 | 0.000 | 0.000 | 0.000 | 0.056 | 0.108 | 0.000 |
| Molar | (a)/(b) | 0.000 | 0.000 | 0.000 | 0.059 | 0.059 | 0.065 | 0.500 |
| ratio of | | | | | | | | |
| functional | (a)/(c) | 0.000 | 0.000 | 0.000 | 0.111 | 0.111 | 0.111 | 0.400 |
| groups | | | | | | | | |

The products obtained have the following characteristics:

TABLE 3

| | Comp. | Comp. | Comp. | | | | Comp. |
|---|---|---|---|---|---|---|---|
| Properties | ex. 1 | ex. 2 | ex. 3 | Ex. 1 | Ex. 2 | Ex. 3 | ex. 4 |
| Content of | 95.3 | 53 | 71.7 | 69.7 | 69.4 | 49.8 | 9.4 |
| THEICT(M)A | | | | | | | |
| (% by weight) | | | | | | | |
| Acrylate | 3.00 | 2.55 | 2.77 | 3.12 | 3.12 | 2.85 | 3.67 |
| functionality (double | | | | | | | |
| bond equiv./mole) | | | | | | | |
| Coloration (APHA) | >120 | 30 | 35 | 30 | 40 | 15 | 70 |
| Viscosity at 50° C. | 900-1100 | 630 | 730 | 2900 | 2660 | 1800 | 6500 |
| (mPa · s) | | | | | | | |
| Recrystallization | 5 | 2 | 3 | 3 | 3 | 0 | N.D. |
| index (0-5) | | | | | | | |
| Ta (° C.) | 265 | 164 | 180 | 202 | 192 | 148 | 39 |

### Compositions Comprising a Mixture of Isocyanurates

Compositions F1-F6 are prepared by combining a mixture of isocyanurates as described above with a photoinitiator at 20° C. (the amounts are stated in parts by weight in the table below).

TABLE 4

| | F1 | F2 | F3 | F4 | F5 | F6 | F6 |
|---|---|---|---|---|---|---|---|
| Product | (comp.) | (comp.) | (comp.) | (inventive) | (inventive) | (inventive) | (comp.) |
| SpeedCure 73 | 4 | 4 | 4 | 4 | 4 | 4 | 4 |
| Comp. ex. 1 | 96 | — | — | — | — | — | — |
| Comp. ex. 2 | — | 96 | — | — | — | — | — |
| Comp. ex. 3 | — | — | 96 | — | — | — | — |
| Ex. 1 | — | — | — | 96 | — | — | — |
| Ex. 2 | — | — | — | — | 96 | — | — |
| Ex. 3 | — | — | — | — | — | 96 | — |
| Comp. ex. 4 | — | — | — | — | — | — | 96 |

The applicative properties of the compositions are detailed in the table below:

TABLE 5

| | | F1 | F2 | F3 | F4 | F5 | F6 | F6 |
|---|---|---|---|---|---|---|---|---|
| Properties | | (comp.) | (comp.) | (comp.) | (inventive) | (inventive) | (inventive) | (comp.) |
| Reactivity (m/min) | | 35 | 5 | 15 | 30 | 35 | 15 | 5 |
| Flexibility (mm) | | >32 | >32 | >32 | >32 | >32 | >32 | <2 |
| Hardness (s) | | 356 | 342 | 347 | 353 | 358 | 357 | 79 |
| Acetone resistance | | 300 | 300 | 300 | 300 | 300 | 300 | 300 |
| (s) | | | | | | | | |
| Stain | Coffee | 0 | 0 | 0 | 0 | 0 | 0 | 3 |
| resistance | Perfume | 0 | 0 | 0 | 0 | 0 | 0 | 0 |
| (0-5) | Iodine | 1 | 2 | 1 | 0-1 | 0-1 | 2 | 5 |

It is immediately clear that comparative example F6 (as described in JP94081782) does not fall within the scope of the invention. The content of THEICTA serving as reactive diluent in the polyester acrylate is much lower (<10%) than that of the other examples (>50%), resulting in much higher viscosity (>6 Pa·s, by comparison with the other values, which are all <3 Pa·s) and a much lower Tα (>40° C., by comparison with the other values, which are all >140° C.).

The use of cekanoic acid in comparative formulations F2-F3 for the esterification of THEIC improves the recrystallization index, but its place in the final ester composition is at the expense of acrylic acid (this is demonstrated by the decrease in the average functionality, which is 3.00 double bond equivalents/mole for THEICTA, but only 2.77 and 2.55 double bond equivalents/mole respectively for the comparative formulations F3 and F2). This difference in structure explains the very sharp decrease in Ta and in hardness, which are key properties of THEICTA.

Formulations F4-F6 comprising the mixture of isocyanurates of the invention demonstrate that the combined use of monoacid and diacids makes it possible to achieve a trade-off in properties (Ta, viscosity, reactivity), while eliminating the problem of recrystallization of THEICTA.

1. A mixture of (meth)acrylated isocyanurates obtained by reaction between:
(a) at least one dicarboxylic acid,
(b) at least one (meth)acrylic monomer selected from acrylic acid, methacrylic acid, anhydrides thereof, and mixtures thereof, and optionally at least one C₆-C₂₄ monocarboxylic acid,
(c) at least one tris(hydroxyalkyl) isocyanurate and optionally another polyol, wherein the molar ratio of —COOH groups of constituent (a) to —OH groups of constituent (c) is between 1:4 and 1:20.

2. The mixture as claimed in claim 1, wherein the tris(hydroxyalkyl) isocyanurate is selected from tris(2-hydroxymethyl) isocyanurate, tris(2-hydroxyethyl) isocyanurate, tris(2-hydroxypropyl) isocyanurate, tris(2-hydroxyisopropyl) isocyanurate, tris(3-hydroxypropyl) isocyanurate, tris(2-hydroxybutyl) isocyanurate, tris(4-hydroxybutyl) isocyanurate, and alkoxylated derivatives thereof.

3. The mixture as claimed in claim 1, wherein the tris(hydroxyalkyl) isocyanurate represents from 75 to 100 mol % of the total number of moles of constituent (c).

4. The mixture as claimed in claim 1, wherein the dicarboxylic acid is selected from:
a saturated aliphatic dicarboxylic acid, selected from the group consisting of adipic acid, sebacic acid, succinic acid, 2-methylsuccinic acid, 2-ethylsuccinic acid, 2,2-dimethylsuccinic acid, 1,11-undecanedioic acid, 1,12-dodecanedioic acid, oxalic acid, malonic acid, 2-methylmalonic acid, 2-ethylmalonic acid, glutaric acid, 3,3-dimethylglutaric acid, 3,3-diethylglutaric acid, pimelic acid, suberic acid, azelaic acid, and a C₃₂-C₃₆ dimer fatty acid;
an unsaturated aliphatic dicarboxylic acid-selected from the group consisting of itaconic acid, citraconic acid, mesaconic acid, glutaconic acid, muconic acid, fumaric acid, or and maleic acid;
a saturated cycloaliphatic dicarboxylic acid selected from the group consisting of cyclopentane-1,2- or -1,3-dicarboxylic acid, cyclohexane-1,2-, -1,3- or -1,4-dicarboxylic acid, cycloheptane-1,2-dicarboxylic acid, and 1,2-, 1,3- or 1,4-bis(carboxymethyl)cyclohexane;
an unsaturated cycloaliphatic dicarboxylic acid; and
an aromatic dicarboxylic acid selected from the group consisting of phthalic acid, isophthalic acid, terephthalic acid, and bis(4-carboxyphenyl)methane;
and derivatives thereof and mixtures thereof.

5. The mixture as claimed in claim 1, wherein the dicarboxylic acid is a a saturated C₄-C₁₀ aliphatic dicarboxylic acid.

6. The mixture as claimed in claim 1, wherein the monocarboxylic acid is present and is selected from:
a saturated monocarboxylic acid such as selected from the group consisting of hexanoic, heptanoic, octanoic, isooctanoic, nonanoic, decanoic, undecanoic, dodecanoic, tridecanoic, tetradecanoic, pentadecanoic, hexadecanoic, heptadecanoic, octadecanoic, 12-hydroxyoctadecanoic, nonadecanoic, eicosanoic, and 14-hydroxyeicosanoic acids;
a monounsaturated monocarboxylic acid such as selected from the group consisting of myristoleic acid, palmitoleic acid, sapienic acid, oleic acid, gadoleic acid, ricinoleic acid, elaidic acid, trans-vaccenic acid, erucic acid, nervonic acid, brassidic acid, and lesquerolic acid; and
a polyunsaturated monocarboxylic acid selected from the group consisting of 7,10,13-hexadecatrienoic acid, 9,12,15-octadecatrienoic acid, 6,9,12,15-octadecatetraenoic acid, 11,14,17-eicosatrienoic acid, 8,11,14,17-eicosatetraenoic acid, 5,8,11,14,17-eicosapentaenoic acid, 6,9,12,15,18-heneicosapentaenoic acid, 7,10,13,16,19-docosapentaenoic acid, 4,7,10,13,16,19-docosahexaenoic acid, 9,12,15,18,21-tetracosapentaenoic acid, 6,9,12,15,18,21-tetracosahexaenoic acid, 9,12-octadecadienoic acid, 6,9,12-octadecatrienoic acid, 11,14-eicosadienoic acid, 8,11,14-eicosatrienoic acid, 5,8,11,14-eicosatetraenoic acid, 13,16-docosadienoic acid, 7,10,13,16-docosatetraenoic acid, 4,7,10,13,16-docosapentaenoic acid, 9,12,15,18-tetracosatetraenoic acid, and 6,9,12,15,18-tetracosapentaenoic acid;
and mixtures thereof.

7. The mixture as claimed in claim 1, wherein constituent (b) comprises a monocarboxylic acid in a molar ratio of monocarboxylic acid to (meth)acrylic monomer of between 5:95 and 15:85.

8. The mixture as claimed in claim 1, said other wherein the another polyol is present and is selected from the group consisting of ethylene glycol, propane-1,2- or -1,3-diol, butane-1,2-, -1,3-2,3- or -1,4-diol, pentane-1,5-diol, hexane-1,6-diol, 3-methylpentane-1,5-diol, decane-1,10-diol, dodecane-1,12-diol, di-, tri- or polyethylene glycol, di-, tri- or polypropylene glycol, cyclohexane-1,4-dimethanol, cyclohexane-1,6-dimethanol, cyclohexane-1,4-diol, bisphenol A, hydrogenated bisphenol A, glycerol, diglycerol, tricyclodecanedimethanol, trimethylolpropane, di(trimethylolpropane), trimethylolethane, hexane-1,2,6-triol, butane-1,2,4-triol, erythritol, pentaerythritol, di(pentaerythritol), neopentyl glycol, 2-butyl-2-ethylpropane-1,3-diol, 2-methylpropane-1,3-diol, 2-methylpropane-1,2-diol, sorbitol, mannitol, xylitol, isosorbide, isoidide, isomannide, methylglucoside, a polyester polyol, a polycarbonate polyol, a polyorganosiloxane polyol, a polyglycerol, a hydroxy-terminated polybutadiene, a diol derived from a hydrogenated or non-hydrogenated dimer or trimer fatty acid, and alkoxylated derivatives of the polyols recited above, and mixtures thereof.

9. The mixture as claimed in claim 1, wherein the reaction mixture does not contain any polyol other than tris(hydroxyalkyl) isocyanurate.

10. The mixture as claimed in claim 1, comprising tris(hydroxyalkyl) isocyanurate triacrylate.

11. The mixture as claimed in claim 1, comprising a polyester component PE comprising a mixture of compounds corresponding to formula (V) below: 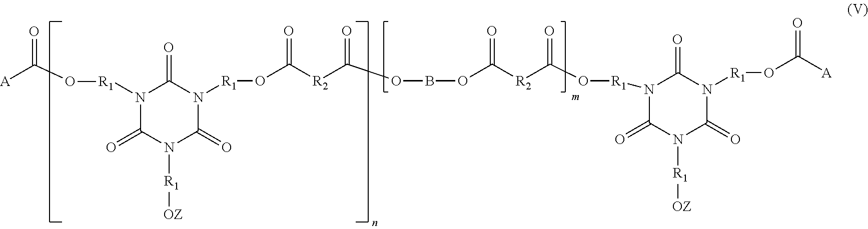
in which:
each R₁ is independently an optionally alkoxylated C₂-C₁₂ alkylene;
each R₂ is independently a dicarboxylic acid residue;
each A is independently a (meth)acrylic acid residue or a monocarboxylic acid residue;
each B is independently the residue of a polyol other than a tris(hydroxyalkyl) isocyanurate;
each Z is independently H or —C(═O)-A;
m and n are average values where n ranges from 1 to 10, m ranges from 0 to 10.

12. The mixture as claimed in claim 1, comprising a polyester component PE comprising a mixture of compounds corresponding to formula (VI) below: 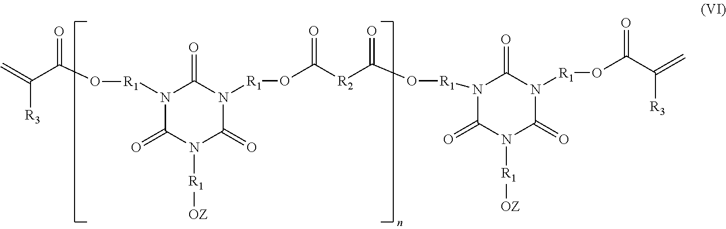
in which:
each R₁ is independently an optionally alkoxylated C₂-C₁₂ alkylene;
each R₂ is independently a dicarboxylic acid residue;
each R₃ is independently H or methyl;
each Z is independently H or —C(═O)—C(R₃)═CH₂;
n ranges from 1 to 10.

13. A process for preparing a mixture of (meth)acrylated isocyanurates as claimed in claim 1, comprising the following steps:
1) reacting all or part of a constituent (b) comprising at least one (meth)acrylic monomer selected from acrylic acid, methacrylic acid, anhydrides thereof, and mixtures thereof and optionally at least one C₆-C₂₄ monocarboxylic acid with a constituent (c) comprising at least one tris(hydroxyalkyl) isocyanurate and optionally another polyol P_{OH} under conditions that permit the esterification of from 50 to 95%, of the OH groups of constituent (c),
2) reacting the mixture from step 1) with a constituent (a) comprising at least one dicarboxylic acid and optionally with any residual amount of constituent (b).

14. A polymerizable composition comprising a mixture of (meth)acrylated isocyanurates as claimed in claim 1 and at least one other ethylenically unsaturated compound.

15. The polymerizable composition as claimed in claim 14, wherein the polymerizable composition is an ink composition, a coating composition, a material filled with fibrous or particulate reinforcements an adhesive composition, molding composition, ink plate composition, electrode binder composition, or a composition for additive manufacturing.

16. A process for making a three-dimensional article by 3D or 4D printing comprising exposing to radiation a polymerizable composition comprising the mixture of (meth)acrylated isocyanurates as claimed in claim 1.

17. The mixture as claimed in claim 1, wherein the dicarboxylic acid is a mixture of succinic acid and sebacic acid.

18. The mixture as claimed in a claim 1, wherein the another polyol is present and is sorbitol.

19. The polymerizable composition as claimed in claim 14, wherein the other ethylenically unsaturated compound comprises a (meth)acrylate functionalized monomer.

20. The mixture as claimed in claim 1, wherein the molar ratio of —COOH groups of constituent (a) to —OH groups of constituent (c) is between 1:6 and 1:12.
